(19) United States
(12) Patent Application Publication
(10) Pub. No.: US 2017/0072175 A1
(43) Pub. Date: Mar. 16, 2017

(54) Dermatology Treatment Device

(71) Applicants: Stanley Batiste, Granite Bay, CA (US); Maryann Batiste, Granite Bay, CA (US); Daniel Batiste, Granite Bay, CA (US)

(72) Inventors: Stanley Batiste, Granite Bay, CA (US); Maryann Batiste, Granite Bay, CA (US); Daniel Batiste, Granite Bay, CA (US)

(21) Appl. No.: 15/260,219

(22) Filed: Sep. 8, 2016

Related U.S. Application Data
(60) Provisional application No. 62/215,639, filed on Sep. 8, 2015.

(51) Int. Cl. A61M 35/00

(52) U.S. Cl. CPC A61M 35/003; A61M 2210/04

(57) ABSTRACT

The device includes a handle configured to be grasped by a user. The handle has a proximal end nearest the user's hand and a distal end opposing the proximal end. The device also includes an applicator with an applicator tip at the distal end of the handle. The applicator tip fits within the depression formed by a skin wrinkle to apply treatment to at least one side wall and bottom area of the wrinkle without applying treatment to surface skin around the wrinkle. The applicator tip may be pre-medicated with medicine, applied to the tip by a user, or provided medication from a chamber in communication with the applicator tip such that the chamber holds medication. Also disclosed is a method for applying wrinkle treatment that includes aligning the applicator tip with the wrinkle and moving the applicator tip along the length of the wrinkle to apply treatment.

### PRIORITY CLAIM

This application claims priority to and the benefit of U.S. Provisional Application 62/215,639 filed on Sep. 8, 2015 entitled Dermatology Treatment Device.

### BACKGROUND OF THE INVENTION

Field of Invention

The present invention relates to treatment of facial wrinkles and in particular to a method and apparatus for applying treatment directly to the wrinkle.

Description of Related Art

It is well known that wrinkles occur as a result of the aging process in people, or due to strain, stress, or repeated facial expressions. Most often, wrinkles form as a part of the natural aging process with factors such as sun exposure, smoking and genetics accelerate their formation. For ages people have tried to both slow the formation of wrinkles and work to eliminate those that already exist. Preventative care with avoidance of the above factors, healthy diet, oral medications and the use of topical medications have been, and are all currently being used. Additionally, Botulism toxin, commonly referred to as Botox is used to slow wrinkle development. Methods to eliminate wrinkles range from using creams and lotions, facial peels to laser treatment and plastic surgery.

There are currently several numerous methods for treating wrinkles once they have formed such as; creams and topical lotions, laser and chemical skin peels, subcutaneous medication injections and surgery. For this invention the injections and surgery are not related art in their application or function although the outcome desired and often achieved is similar. The invention however does have some relative similarities to the function of laser and chemical treatments and, although less so, the topical application properties. Chemical peels and lasers kill layers of cells, and depending on the applications, can involve the superficial to deep skin levels. Once the cells die they are rapidly replaced by regenerative process of cell and support tissue growth, basically, creating new skin surface. This is generally a diffuse application process whether for the treatment of a specific wrinkle or large areas of skin. Many medications also create cell and support tissue growth, one of which is retinol. Retinol is a form of vitamin A that is able to penetrate to the lower layers of skin where it helps rebuild collagen and elastin, which is why it has been shown to be helpful. Retinol is applied usually to a wide area but can be placed focally over smaller areas of interest.

Currently, there are many good means to slow the formation of wrinkles and decrease their appearance as well as completely remove them. All of the current methods however are limited in either their effectiveness or lack the wrinkle specific focus or both. The lack of effectiveness is noted in many of the creams and lotions that are applied topically over and ultimately within the wrinkle. These creams, such as retinol can cause cell and support tissue growth. However, this has been shown to be limited when compared to stronger means such as chemical peels and laser. Chemical peels and laser have shown to be very effective in creating cell death and skin turnover up to very deep skin layers. Although the method is proven, the problem is that the death of non-wrinkled tissue, as well as the wrinkle serves as the reparative mechanism. This method then utilizes a means of wrinkle repair that makes broad strokes and is applied to undamaged adjacent skin surfaces. This is most notable with lasers, although very effective, they do not extend to the wrinkle but involve areas of adjacent tissues.

Plastic surgery also is quite effective in eliminating wrinkles as it increases shear stresses, literally unfolding the wrinkle through stretching the skin. Although very effective, this method requires an invasive procedure with healing time and carries the risk of numerous complications. Surgery is also the most expensive of the options generally costing thousands of dollars although it remains the most effective solution.

### SUMMARY

To overcome the drawbacks of the prior art and provide additional benefit, a device for applying medication to a wrinkle is disclosed. The proposed new invention utilizes a combination of a uniquely designed applicator and medications to focus directly on the wrinkle, specifically the walls of the skin lining the wrinkle and the acute angle at its furthest distance from the skin surface.

In one embodiment, this device includes a handle configured to be grasped by a hand of a user. The handle has a proximal end nearest the user's hand and a distal end opposing the proximal end. The device also includes an applicator attached to or located at the distal end of the handle. The applicator has an applicator tip configured to fit within the depression formed by a skin wrinkle to apply treatment to at least one side wall and bottom area of the wrinkle without applying treatment to surface skin around the wrinkle.

In one embodiment, the treatment is medication. In another embodiment the treatment type is selected from the following group of treatment types: thermal treatment, light or laser treatment, ultrasonic treatment, and radio frequency treatment. It is also contemplated that the device may further comprise a second applicator tip at the proximal end of the handle such that the second applicator tip is configured to fit within the depression formed by a skin wrinkle to apply treatment to the wrinkle. The device may further comprise a chamber configured to contain medication such that the chamber is located in the handle or near the applicator and having an opening which leads to the applicator tip to supply the medication to the applicator tip during use.

The applicator may be a roller having a center that is secured to the distal end of the handle while allowing rotation of the roller and the applicator tip is the outer edge of the roller. In one embodiment the applicator tip emits light energy generated by a signal generator, the signal generator supplied with power from one or more batteries stored in the handle.

Also disclosed is a method for applying wrinkle treatment to a skin winkle that has a length and side walls that form depression having a bottom compared to surface skin on each side of the wrinkle. This method comprises opening a package containing an applicator such that the applicator has a handle section and an applicator tip. Then removing the applicator from the package by the handle and aligning the applicator tip with the wrinkle. Once aligned, moving the applicator tip into the depression of the wrinkle and moving the applicator tip along the length of the wrinkle while the applicator tip is in contact with the side walls and bottom of the wrinkle. This applies treatment to the side walls and bottom but not the surface skin on each side of the wrinkle.

In one embodiment, the applicator tip is premeditated with wrinkle treatment. In another variation the method operation further comprising dispensing medication from a chamber through a medication pathway to the applicator tip. This method may also include washing the skin and skin wrinkle prior to moving the applicator tip along the length of the wrinkle. It is contemplated that the applicator tip is an outer edge of a roller and the roller is connected to the handle at the center of the roller while allowing rotation of the roller in relation to the handle. This method of operation may further comprise switching on the applicator to activate treatment therapy prior to moving the applicator tip along the length of the wrinkle such that the treatment therapy is emitted from the applicator tip into the depression of the wrinkle. The treatment therapy comprising one of the following: thermal treatment, light or laser treatment, ultrasonic treatment, and radio frequency treatment.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The components in the figures are not necessarily to scale, emphasis instead being placed upon illustrating the principles of the invention. In the figures, like reference numerals designate corresponding parts throughout the different views.

FIG. 1 illustrates an exemplary facial wrinkle with close up view.

FIG. 2 illustrates the three primary layers of the skin.

FIG. 3 illustrates an exemplary applicator showing the handle, roller and axle.

FIG. 4 illustrates and assembly drawing of the applicator.

FIGS. 5A, 5B, 5C, 5D, and 5E illustrate various applicator roller heads.

FIG. 6 illustrates the applicator being used to treat a facial wrinkle.

FIG. 7 illustrates the applicator in alignment with a wrinkle.

FIGS. 8, 9 and 10 illustrate the applicator in use within a wrinkle.

FIG. 11 illustrates a cross section of a wrinkle before and after therapy application.

FIG. 12 illustrates an applicator with internal power supply and associated therapy unit.

FIG. 13 illustrates an exemplary method of use of the applicator.

FIG. 14 illustrates an applicator in use applying emitting of therapy to a patient.

FIG. 15 illustrates an example embodiment of a thin tip applicator.

FIG. 16 illustrates an exemplary flat applicator.

FIG. 17 illustrates the exemplary flat applicator in use on a patient.

FIG. 18 illustrates an example embodiment of a pencil type applicator

FIG. 19 illustrates the exemplary pencil type applicator in use.

FIG. 20 illustrates an exemplary pencil type applicator emitting wrinkle reduction therapy.

FIG. 21 illustrates a cross section of wrinkles pre and post therapy and with regions of skin effected by the therapy.

### DESCRIPTION OF THE INVENTION

Disclosed herein is a new type of pharmacological, light therapy, radiotherapy, or thermal applicator designed to treat wrinkles by reducing wrinkle formation, depth, noticeability, and appearance. As shown in FIG. 1, the anatomy of a wrinkle 104 on a patient 100 is like that of a very steep valley with side walls 108 which come together at the lowest level forming a bottom 112. On each side of the wrinkle 104 is surface skin 120. Depending on numerous development factors, including skin integrity, wrinkles can be anywhere from less than a millimeter to several millimeters in depth. Wrinkles can occur everywhere in our skin with the most common locations along areas of increased stress and excess skin growth, or areas of skin damage.

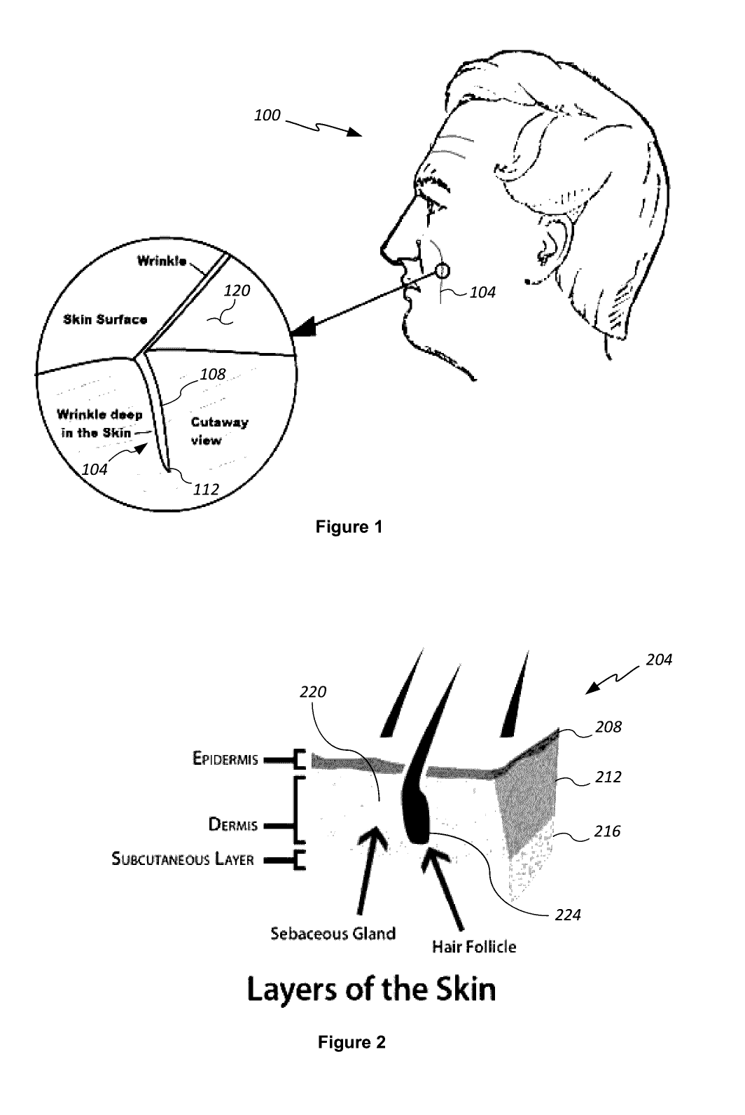

As shown in FIG. 2, skin 204 is composed of three layers: the epidermis 208, the dermis 212, and the subcutaneous fat 216. The walls 108 (FIG. 1) of the wrinkle 104 are composed of epidermis 208 which protrudes in the dermis 212. The innovation involves a means to treat the walls 108 and bottom 112 of the wrinkle 104 by directly applying medications without application to the surrounding, normal skin surface 120.

Currently, the method for treating wrinkles involves topical medications, lasers and surgery. Topical medications are usually applied to the local skin area 120 surrounding the wrinkle. Ideally, this medication would be pushed into the valley of the wrinkle 108 in it application. However, most often the user's generally flat or oval palm or finger does not place the medication into the valley the wrinkle, but only on the top surface 120 of the skin. As to laser light, the side walls 108 receive very little treatment light, much like a steep valley in nature receives only limited and diffused sunlight. Any laser light that does project downward into the wrinkle 104 strikes the side walls 108 at a glancing angle thereby providing very little treatment energy. Another drawback to the prior art is that using this process the medication is also applied to skin surrounding the wrinkle. Any side effects of medication application to normal skin is also seen with the use of lasers as well as chemical peels with surgery also affecting the surrounding skin. The surface skin 120 may not need treatment and such treatment may actually damage or otherwise affect the skin in an unwanted way.

This new innovation allows the user to apply therapy, whether pharmacological, phototherapy, radiotherapy, ultrasonic or thermal directly in the valley of the wrinkle without involving the adjacent skin surface. This application devices and method of use protects the adjacent skin and therefore creates less of an unpleasant cosmetic appearance during and after therapy, which is caused by treatment to the surface skin 120. This means of application also results in more direct, intense therapy that can be applied thereby decreasing the applications needed and improving the efficacy of each application. The net result of this is reduced costs or a better outcome for a given treatment cost due to more treatment per dollar spent.

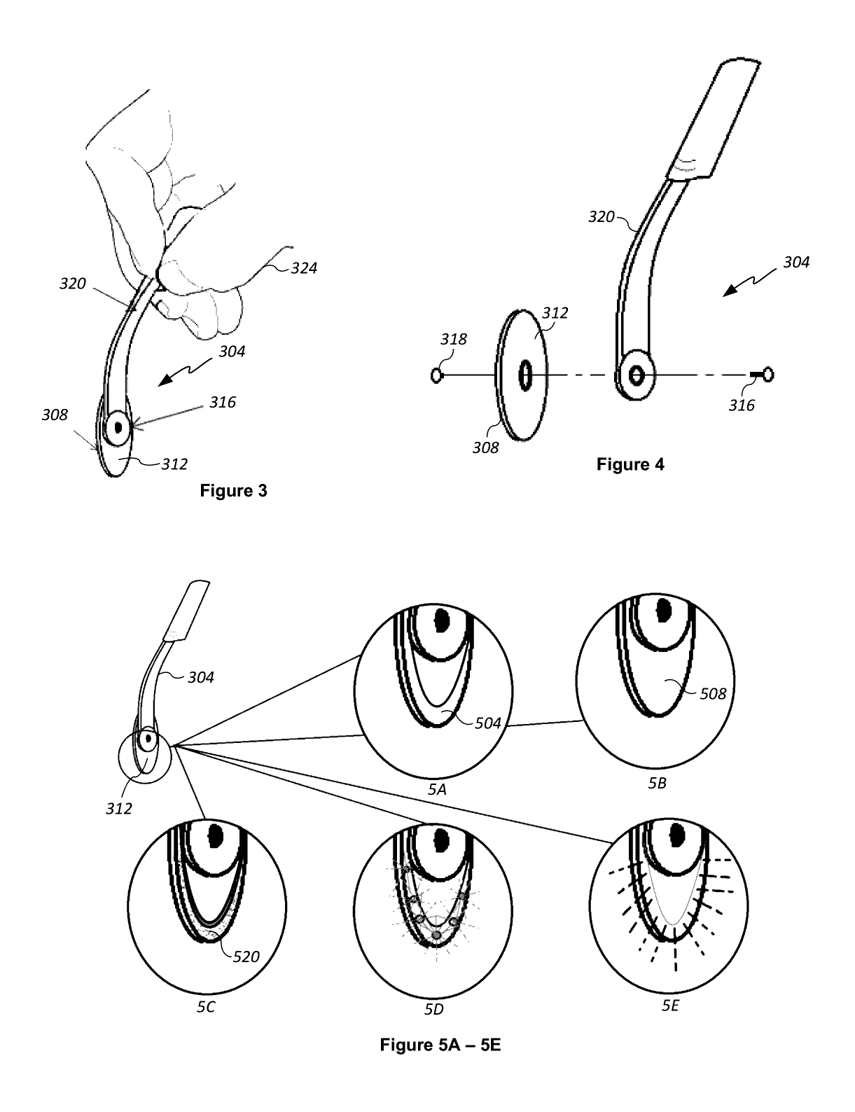

The design disclosed herein utilizes an applicator 304 which may assume many different configurations. In this example embodiment a thin applicator head 308 is configured to fit within and travel within the wrinkle opposing the valley walls 108 and the bottom simultaneously 112. In the first embodiment as shown in FIG. 3, the applicator 304 has a narrow roller 312 that is attached by means of an axle 316 or other mechanism to support the roller 312. In this embodiment, the roller 312 has a narrow outer head 308 that can fit into the depression formed by the wrinkle. A handle 320 as shown is connected to the roller by the axle 316. The axle may be secured with a pin, ring 318, or other element to maintain the roller 312 connected to the handle 320. A user may use their hand 324 to grasp the handle 320 in use to direct the narrow head 308 into the wrinkle valley. FIG. 4 illustrates an exploded and enlarged view of the applicator 304 and identical elements are referenced with identical reference numbers.

Pharmacological, light therapy, radiotherapy, ultrasonic or thermal materials are present of the outer surface of the wheel, the narrow head 308, or both as shown in FIGS. 5A-5E. As compared to FIGS. 3 and 4, similar elements are identified with similar reference numbers. In FIG. 5A the treatment material is applied directly to a solid or porous wheel 504. In FIG. 5B, the treatment material is applied to an absorbent material such as a thin sponge 508 or other applicator material that will hold wrinkle reducing treatment material during application. FIG. 5C illustrates a thermal applicator ring 520. Thermal applicators may include but are not limited to any mechanism that applies heat, with or without medication, the wrinkle depression. It is contemplated that the user of thermal energy for treatment, such as but not limited to radio frequency energy, may be used alone or in connection with medication being applied to or previously applied to the wrinkle.

FIG. 5D illustrates an applicator configured for radiotherapy conductor, ultrasonic therapy, or light source in phototherapy in which the disk 312 is configured to emit light (any wavelength) or ultrasonic therapy to the sides and downward. In the case of pharmacological materials, it can make up the entire composition of the wheel as shown in FIG. 5E.

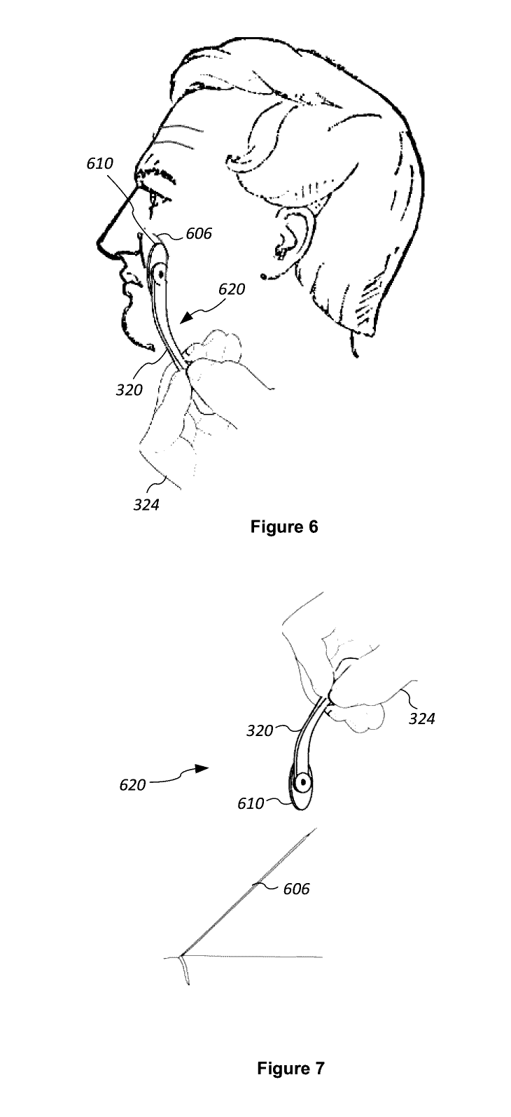

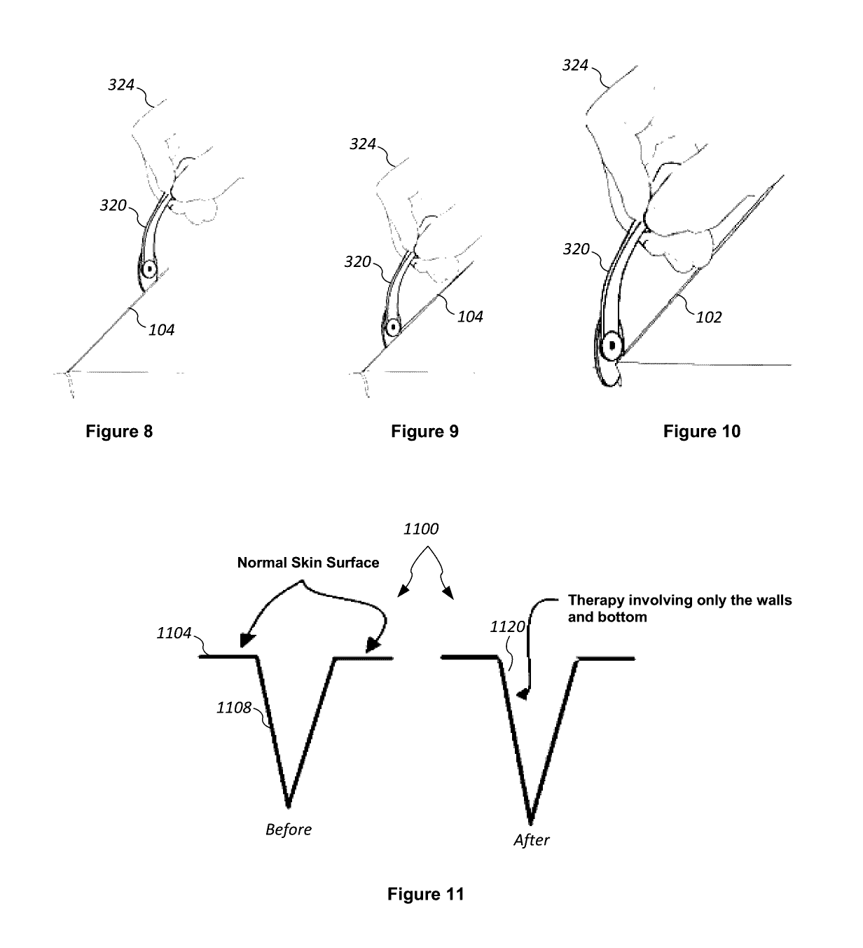

FIGS. 6 and 7 illustrate exemplary methods of use of the applicator. To operate, the user 324 holds the handle 320 and aligns it directly into the patient's wrinkle 606 such that the narrow outer edge 610 of the applicator is inserted inward toward the wrinkle. The roller applicator 620 is then rolled along the length of the wrinkle as shown in FIGS. 8, 9, and 10 from one end of the wrinkle to the other end of the wrinkle. By moving the applicator along the valley formed by the wrinkle, medication coats the valley walls and bottom of the wrinkle with the means of therapy. In other embodiments, treatment therapy such as light, heat, or ultrasonic will be applied to the wrinkle in the same manner. It is also contemplated that as part of this treatment method the person applying the wrinkle therapy may spread the skin apart in the area of the wrinkle to further expose the sides and bottom of the wrinkle. In one embodiment, the applicator includes a spreader, such as one or more bars or feet that spread the wrinkle as the applicator tip is run down the wrinkle. Also part of the method of use, the user may first wash the face to open the pores, clean the area of the wrinkle, and have a moist face such that the carrier may act as a carrier of the treatment medication.

FIG. 11 illustrates the before and after views of a wrinkle. As shown, the surface skin 1104 is above and on each side of the wrinkle 1100. It may have a generally normal appearance and not require treatment. In fact, treatment to this surface skin area 1104 may be harmful and unwanted. The wrinkle 1100 also includes side walls 1108 which form the wrinkle as a depression in relation to the surface skin 1104. As shown in the after figures, the side walls 1108 are coated in medication 11209 or received treatment from the applicator. The effect of this treatment is wrinkle reduction. In use, the roller application of treatment therapy (medication or other treatment) may be applied several times, as needed.

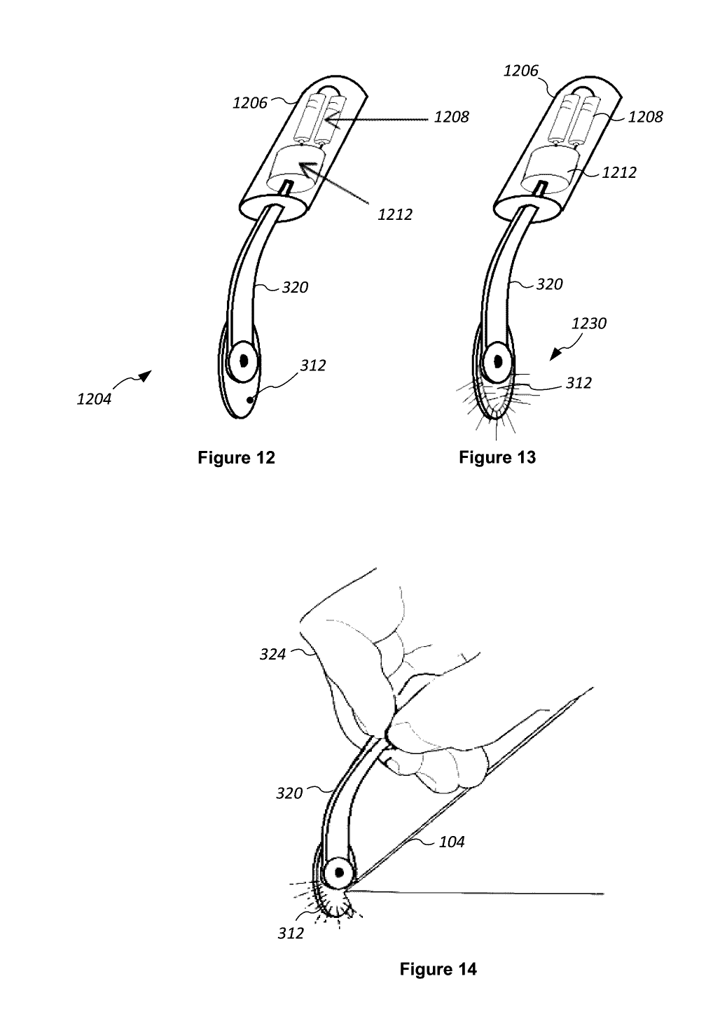

FIG. 12 illustrates an example embodiment of an applicator utilizing thermal, radiofrequency, ultrasonic therapy or light source therapy. In this example embodiment the applicator 1204 includes a power source 1208 such as batteries contained within housing 1206 or other chamber. In other embodiments, the power source may be hard wired power from an electrical plug in. Also shown within the housing 1106 is the therapy generation means, such as light source or other source of treatment. This device 1204 may then be transfer the therapy down the handle 320 to the therapy roller 1230 causing the treatment to extend outward as shown in FIG. 13 from the applicator or tip. FIG. 13 illustrates the applicator 1204 in operation with the therapy as being emitted 1230 from the wheel 312 toward the sides and/or bottom of wrinkle.

In reference to FIGS. 12 and 14, it is also contemplated that the therapy generating elements may be located in the applicator portion 312. Light emitting elements may be made small, or the wheel portion may be made of an optic conducting elements, which direct the light toward the edges, such as for example with reflective mirrors on the sides which direct the light downward to the outer edge and down into the wrinkle. The source of the therapy may also be in any other portion of the device and directed to the applicator tip or narrow edge. Other means to provide the energy source such as external power including standard AC current or with gas cartilages are not shown but are contemplated to operate the treatment means for the applicator.

FIG. 14 depicts the applicator with treatment shown within the wrinkle providing treatment, such as using thermal, radiofrequency, ultrasonic therapy or light source therapy. Use of the embodiment shown in FIG. 14 would be generally similar to the method of operation described in connection with FIGS. 8-11.

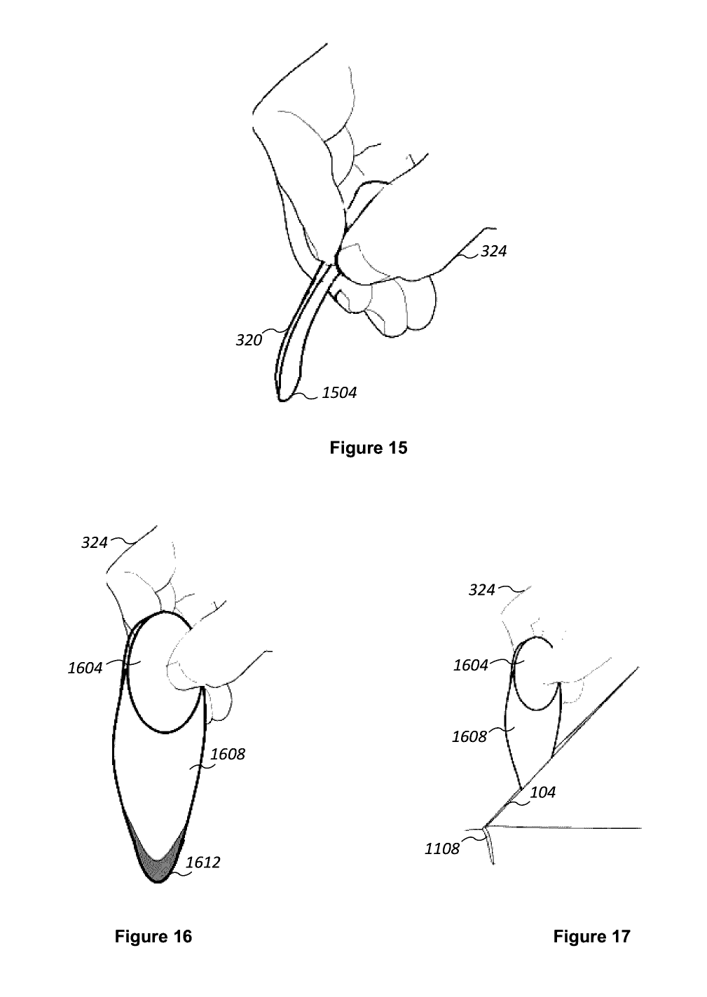

FIG. 15 illustrates an alternative embodiment of the applicator. In this embodiment the roller may be smaller and the handle may also be smaller, such as having a thin tipped handle 320. It is also contemplated that the roller may not roll, but be fixed in place. Or, only a portion of the roller may be provided, such as shown in FIG. 15 with a semi-circular shape 1504 that is provided with medication (pre-medicated or applied at time of use).

FIG. 16 illustrates another embodiment of the applicator. In this embodiment a user 324 grasps the generally oval handle 1604 that connects to a body 1608. Opposite the handle 1604 is an applicator tip 1612 configured as a thin surface. Any treatment method may be applied to, pre-applied to, or emitted from the applicator tip 1612. The method of use associated with the embodiment of FIG. 16 is generally similar to the method of use and operation for the embodiment shown in FIGS. 8-11 to engage the inner portion of the wrinkle 1108, as shown in FIG. 17, to provide treatment to generally only the inner sides and bottom of the wrinkle. The applicator tip 1612, much like the roller applicator, can use a variety of the means for treatment application as described for the roller applicator. For example, in the case of a light therapy device, button batteries may be used in the thin handle and the light therapy directed down through the body 1608 to the applicator tip.

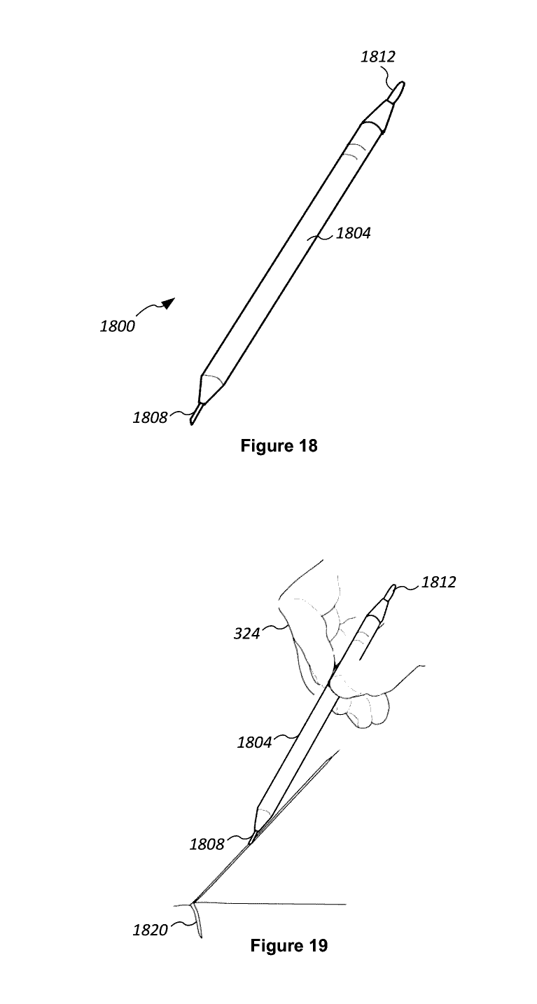

FIG. 18 illustrates yet another embodiment having an elongated configuration. In this embodiment, a pencil type applicator 1800 has a thin elongated body 1804 which has a first tip 1808 and a second tip 1812 on opposing ends of the body. The first tip 1808 and the second tip 1812 may be the same, configured in different shapes or sizes, or configured to provide different types of treatment. As shown in FIG. 19, the tip 1808 is configured to fit into the wrinkle 1820 to apply any type of treatment discussed herein or developed in the future that engages the inner portion of the wrinkle 1820 with the same application means as the roller applicator described above.

In one embodiment, the pencil applicator uses an adjustable tip 1808, 1812 that can be continually lengthened or shortened to supply the treatment materials. The tips 1808, 1812 can be any type shape or arrangement such that the tip is generally narrow and thus able to apply treatment to the inner sides and/or bottom of a wrinkle 1820. The tips 1808, 1812 may be made of any type material. For example, the tips 1808, 1812 may be smooth to allow the tip to easily slide along the wrinkle, or the tips may be porous to better hold treatment material and apply the treatment material to the wrinkle. The tips may be metal which are good at conducting heat and cold, or plastic, or porous, such as a felt tipped marker material. In any of the embodiments described herein, the term narrow is defined as being able to fit within a wrinkle to apply medication or treatment to the wrinkle. In one embodiment the term narrow is defined as less than ½ inch. In another embodiment the term narrow is defined as less than ¼ inch. In another embodiment the term narrow is defined as less than ⅛ inch. In another embodiment the term narrow is defined as less than 1/16 inch.

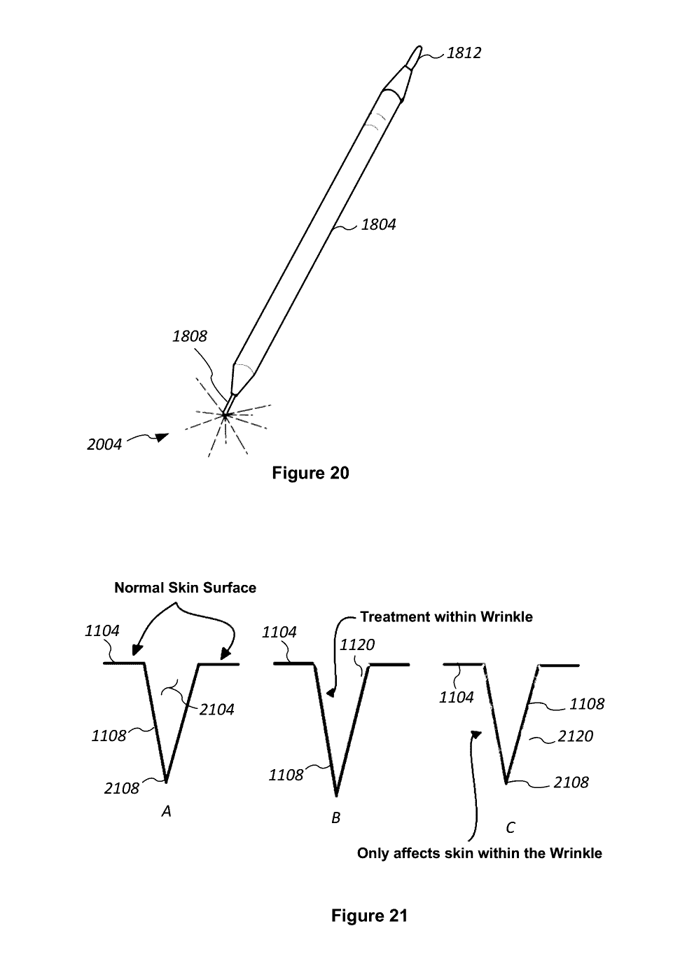

In an alternative embodiment of the pencil type applicator 1800, the thin tip 1808 houses the means to produce light therapy, radiotherapy, thermotherapy and ultrasonic therapy among the others previously mentioned treatment types. The light or other type therapy 2004 is emitted from one or more of the tips 1808, 1812. This embodiment is shown in FIG. 20. The body 1804 may include a battery, control electronics, one or more switches or power control (user interface) and a therapy source directed to the tip 1808.

FIG. 21 illustrates the resulting treatment area using the applicator system described herein. As shown in view A, the wrinkle 2104 is formed by a depression in the surface skin 1104. The depression is formed by side walls 1108 which terminate at a bottom 2108.

As shown in view B, with all the applicators described herein the treatment materials or therapies 1120 are applied only on the walls 1108 and the bottom of the wrinkle, keeping the surface skin 1104 free from treatment 1120. This function will allow the use of stronger materials and/or thermal gradients without damaging normal skin. As shown in view C, the treatment penetrates 2120 the skin in the wrinkle area without treating the other areas on the surface skin 1104. The materials used will vary in the number of applications needed and the effect on the wrinkled tissues which will in turn vary the therapeutic benefit derived from treatment.

It is contemplated that if the treatment being applied to the wrinkle is medication, such as liquid, cream, gel, or other medicine, then the applicator may be pre-medicated. In such a configuration, the applicator tip or portion that fits into the wrinkled area has medication pre-applied and the applicator may be disposable after one or more uses. The applicator tip may be porous or absorbing to hold medication sufficient for one or more uses. The entire applicator may be sealed in a sealed foil wrap.

It is also contemplated that the application may be dipped into medication or have medication applied to the applicator portion that fits into the wrinkle. The device could be reusable or disposable. Any type medication cream, gel, or liquid may be used.

It is further contemplated that the housing or other part of the applicator may include a chamber which stores medication. The chamber includes a passage way to the applicator tip. During use, the medication in the chamber is provided to the applicator tip and then applied to the wrinkle. The medication stored in the chamber of the application may be provided to the tip in any manner known in the art including through capillary action, by gravity, through internal pressure, such as squeezing, a plunger, or other means. The applicator with medication storage chamber may be sold as a complete unit or the user may fill the applicator chamber with the medication best suited for treatment. In this embodiment with a chamber holding medication, the user may control the amount of medication applied to the wrinkle and utilize the applicator repeatedly. The chamber may be located in any part of the applicator. For example, in the embodiment of FIG. 4, the chamber may be in any part of the handle 320. In the embodiment of FIGS. 15 and 16, the chamber may be in the handle 320, 1604, or the body 1608 to feed medication to the tip 1504, 1604. In the embodiment of FIG. 18, the body 1804 may be hollow thereby providing or to establish a chamber which provides medication to the tips 1808, 1812 such as by gravity, capillary action, squeezing, twisting of the body, or any other means.

It is also contemplated that two or more types of treatment may be combined such that medication is disposed from the same applicator tip while another type of therapy is being provided, such as thermal, light, ultrasonic, or any other type of treatment. By combining two types of treatment, the effects may be improved. In another embodiment, one tip of the embodiment of FIG. 20 applies medication while the opposing tip applies light, thermal, radio frequency, or ultrasonic treatment.

While various embodiments of the invention have been described, it will be apparent to those of ordinary skill in the art that many more embodiments and implementations are possible that are within the scope of this invention. In addition, the various features, elements, and embodiments described herein may be claimed or combined in any combination or arrangement.

What is claimed is:

1. A device for applying medication to an area inside a wrinkle comprising:
a handle configured to be grasped by a hand of a user, the handle having a proximal end nearest the user's hand and a distal end;
an applicator attached to or located at the distal end of the handle, the applicator having an applicator tip configured to fit within the depression formed by a skin wrinkle to apply treatment to at least one side wall and bottom area of the wrinkle without applying treatment to surface skin around the wrinkle.

2. The device of claim 1 wherein the treatment is medication.

3. The device of claim 1 wherein the treatment is a treatment type selected from the following group of treatment types: thermal treatment, light or laser treatment, ultrasonic treatment, and radio frequency treatment.

4. The device of claim 1 further comprising a second applicator tip at the proximal end of the handle, the second applicator tip configured to fit within the depression formed by a skin wrinkle to apply treatment to the wrinkle.

5. The device of claim 1 further comprising a chamber configured to contain medication, the chamber located in the handle or near the applicator and having an opening which leads to the applicator tip to supply the medication to the applicator tip during use.

6. The device of claim 1 wherein the applicator is a roller having a center that is secured to the distal end of the handle while allowing rotation of the roller and the applicator tip is the outer edge of the roller.

7. The device of claim 1 wherein the applicator tip emits light energy generated by a signal generator, the signal generator supplied with power from one or more batteries stored in the handle.

8. A method for applying wrinkle treatment to a skin winkle that has a length and side walls that form depression with a bottom in relation to surface skin on each side of the wrinkle, the method comprising;
opening a package containing an applicator, the applicator having a handle section and an applicator tip;
removing the applicator from the package by the handle;
aligning the applicator tip with the wrinkle;
moving the applicator tip into the depression of the wrinkle;
moving the applicator tip along the length of the wrinkle while the applicator tip is in contact with the side walls and bottom of the wrinkle to thereby applying treatment to the side walls and bottom but not the surface skin on each side of the wrinkle.

9. The method of claim 8 wherein the applicator tip is premeditated with wrinkle treatment.

10. The method of claim 8 further comprising dispensing medication from a chamber through a medication pathway to the applicator tip.

11. The method of claim 8 further comprising washing the skin and skin wrinkle prior to moving the applicator tip along the length of the wrinkle.

12. The method of claim 8 wherein the applicator tip is an outer edge of a roller, the roller is connected to the handle at the center of the roller while allowing rotation of the roller in relation to the handle.

13. The method of claim 8 further comprising switching on the applicator to activate treatment therapy prior to moving the applicator tip along the length of the wrinkle such that the treatment therapy is emitted from the applicator tip into the depression of the wrinkle, the treatment therapy comprising one of the following: thermal treatment, light or laser treatment, ultrasonic treatment, and radio frequency treatment.
